(19) United States
(12) Patent Application Publication
(10) Pub. No.: US 2022/0008597 A1
(43) Pub. Date: Jan. 13, 2022

(54) Method and System for Verifying the Disinfection of a Confined Space

(71) Applicant: Benjamin Bergman, Florham Park, NJ (US)

(72) Inventor: Benjamin Bergman, Chatham, NJ (US)

(21) Appl. No.: 16/924,325

(22) Filed: Jul. 9, 2020

(51) Int. Cl. A61L 9/14; G06Q 10/02; G01C 21/34; G07C 9/00; G06K 9/00

(52) U.S. Cl. CPC A61L 9/14; G06Q 10/02; G06K 9/00838; G07C 9/00309; G01C 21/3438

(57) ABSTRACT

This invention provides assurance to a party that wishes to rent or occupy a confined space such as a hotel room, short term office space rental, taxi, car service, rental vehicle or other similar confined space that such space has been treated with a germicidal and virus killing treatment since the confined space was last rented out. This germicidal and virus killing treatment is applied after normal housekeeping, in the case of hotel rooms and office space rental, and after car servicing, in the case of car rentals, providing assurance that the confined space has not been touched by human hands after such treatment has been completed.

### CROSS REFERENCE TO RELATED APPLICATIONS

None

### STATEMENT REGARDING FEDERALLY SPONSORED RESEARCH OR DEVELOPMENT

None

### JOINT RESEARCH AGREEMENT

None

### SEQUENCE LISTING

None

### STATEMENT OF PRIOR DISCLOSURES BY INVENTOR

None

### BACKGROUND

The emergence of SARS-CoV-2 (revered to herein as Covid-19) in early 2020 has created extreme pressure on germicidal and virus killing treatment techniques and particularly those used for confined spaces. The approaches currently being used by car rental companies, car services, taxis, hotels, office space rentals and other confined spaces to kill germs and viruses rely on a person to a wipe or spray down the surfaces with a germicidal and virus killing agent and/or allowing enough time to pass to complete an air exchange to remove the remaining germs and viruses. These approaches do nothing to provide actual information to the prospective occupant as to when and how a germicidal and virus killing treatment was applied to the confined space or whether anyone has occupied the space since the treatment was applied.

These same businesses have adopted programs for cleaning processes, testing, and inspection of cleaning processes, but all of them invariably rely on the word of a person, rather than actual data as to the treatment that was applied and when it was applied.

The same is true regardless of whether the treatment consists of a fogger, UV-light, ozone generators, or robots spraying an aerosol. Airlines are increasing cleaning times and using better air filters; United is advertising electrostatic spraying on airplanes. All of these approaches do not address the issue of providing actual information as to the germicidal and virus killing treatment that was applied to the specific confined space that the prospective occupant is waiting to occupy, nor do they provide a method of

securing the confined space after it has been treated with a germicidal and virus killing treatment. Fogging and spraying of disinfectant have been a mainstay of viral decontamination for decades in the hospital and public spheres. In the hospital setting adjunctive methods of airborne transmitted pathogens include negative pressure rooms, UV light, high filtration masks and particulate filters. The public sphere has far fewer pathogen control measures. The modern era has seen transmissible viruses such as influenza remain a substantial pathogenic force, even when a vaccine and drug therapy exist. Our current virus control systems have been overwhelmed by the contagiousness and persistence of Covid-19, for which there is no curative treatment.

None of the systems address the issue of communicating the germicidal and virus killing treatment that was applied, when it was applied, or whether the room has been occupied since it was applied. The coronavirus pandemic has eroded the normal public confidence relying on the word of the people who control the confined spaces such as car services, taxis, hotel rooms, office space rentals, rented automobiles or similar confined spaces that such confined spaces have been appropriately treated after the prior occupancy.

To our knowledge, no complete systems or methods currently exist that take the process from initial confirmation that the space is empty through communication of the treatment and the products used in such treatment to the prospective occupant and securing the confined space until the prospective occupant enters the space. In order to re-establish confidence in sanitization and thereby use of spaces, some companies have hired other companies to perform “Masters of Sanitation” services, which involves entering a space, provide reliable cleaning services while space is observed to be empty with appropriate personal protective equipment.

Such protective equipment consisting of eyewear (such as goggles or face shield, or safety glasses, N95 respirator with activated charcoal filter in many cases, or powered air purifying respirator, gloves, long sleeves, and long pants) is required due to the method of sanitization with a person present, thereafter certifying the sanitization of the space by virtue of their performed services. None of the existing art contains non-human automated “master of sanitation” services.

There is a need for a system that can determine that the room is empty, treats the room with a germicidal and virus killing treatment, confirms that the treatment was completed and sends an electronic communications to the prospective occupant that it has been treated and that only the prospective occupant has been provided with the code to enter the confined space, without the presence of a “Master of Sanitization.” As used in this application, the term electronic communications is intended to include any form of electronic signal whether in text, text message, code, phone message, digital translation or other form that is capable of being understood and acted upon by the devices that comprise the system and are used in the method.

While components used in this system have existed and been the subject of prior issued patents, no one has provided a complete system that provides the prospective occupant waiting to occupy the space with confirmation both of treatment and that no one has occupied the space after the treatment was completed. For example, U.S. Patent Publication 2012/0039752 discloses an air treatment device for use in treating air to remove contaminants or bad odors through the combination of air filters and an air treatment agent. U.S. Pat. No. 8,741,223 discloses a method for applying chlorine dioxide within a confined space for the purpose of eliminating contaminants.

The '223 patent discloses how chlorine dioxide may be used to eliminate contaminants, but not a system that provides assurance to the prospective occupant that such treatment has been completed. U.S. Pat. No. 8,680,496 discloses a system and method for the germicidal sanitizing of elevators or other confined structures. While the same system could be applied to other confined spaces, it does not communicate with the prospective occupant. U.S. Pat. No. 9,498,552 discloses a mist generating device for treating mold, bacteria and odors in vehicles that is powered by the vehicle's low direct current voltage power supply. U.S. Pat. No. 8,872,665 discloses a hygiene compliance

monitoring system which checks that an individual health care worker has complied with the hygiene compliance before seeing a patient who is in a particular hospital (or facility) bed. The first is a patent publication which offers a method for treating air but does not focus on a germicidal and virus killing treatment. While the second, third and fourth disclose a system for the treatment, they do not disclose a complete system for determining that the space is empty, treating the space and confirming to the prospective occupant that it has been treated. While the fourth patent discloses a system for verifying hygiene compliance, that is very different from verifying that a confined space has been treated.

None of these patents address the fundamental issue of communicating the treatment and when it was completed to the prospective occupant to provide assurance that the confined space is free of virus and germs. It is an object of this invention to facilitate the verification that confined indoor spaces have been treated with a germicidal and/or virus killing treatment and therefore are safe (at least from the standpoint of germs and viruses) to be occupied, thus making travel (e.g. car rental, taxis, ride services, hotel rooms, cruise ship rooms and other contained spaces) safer and more appealing to potential travelers

### SUMMARY OF INVENTION

Embodiments of the present invention solve the above mentioned problems and provide a distinct advance in providing customer reassurance that a germicidal and virus killing treatment has been applied to the confined space in question and communicating to the prospective occupant (the “Initiating Party”) that a confined space has been treated and is ready for occupancy. When a prospective Initiating Party approaches a party that controls a confined space (the “Controlling Party”), whether for check in (lodging and auto rental, for example) or to occupy the vehicle (e.g. car service, taxi), the Controlling Party sends an electronic communications to a computer initiating the germicidal and virus killing treatment cycle. In the case of vehicles and lodgings, this could be done by a person at the location where check in occurs or may be done electronically.

If remote electronic check in is used, the completion of the check in process can initiate the germicidal and virus killing treatment. In the case of car services and taxis, the driver would need to exit the vehicle first and then initiate the germicidal and virus killing treatment cycle. If the vehicle has a confined space that is sealed off from the driver's space, then the driver need not exit the vehicle to initiate the treatment process and a locking code could still be given to the Initiating Party that unlocks the passenger's confined space but not the driver's separate space.

Embodiments of the system for disinfecting a confined space comprise one or more sensors controlled by a computer to check the confined space to verify that the space is not occupied. The system further comprises a computer with the ability to accept electronic communications from the sensors and further to send and receive such additional electronic communications as are described herein. Optionally, the computer communicates with door locks to lock the confined space before treatment and sets a locking code that must be used to open the door after treatment which can be communicated to the Initiating Party so that the space can only be entered by the Initiating Party after the treatment has been completed, thus providing assurance to the Initiating Party that the confined space has not been entered since treatment was completed. The computer also communicates with the germicidal and virus killing treatment device in the confined space to start the germicidal and virus killing treatment process. The treatment device may be a fogging system, ultraviolet light system or other systems known to those skilled in the art to be effective at killing germs and viruses in a confined space that is the size and configuration of the confined space that it services. The treatment device has the ability to send an electronic communication to the computer upon completion of the treatment cycle confirming that the treatment cycle has been completed and providing the time of completion.

Optionally, for treatment devices using a material (e.g. fogging or gassing device) the computer may query the treatment device and obtain confirmation of materials used by the treatment device for sterilizing the confined space and the source of those materials. Optionally, for treatment devices consisting of a fogging or gas passing device, the treatment device may include a heating element. Optionally, one or more pumps or compressors may be used by the treatment device to delivery the disinfectant.

Optionally, it may also provide other information about the germicidal and virus killing treatment process that is obtained either from the treatment device or one or more sensors. Upon receipt of confirmation that the treatment cycle has been completed including any time for the treatment material to settle, the computer sends a communication advising the Initiating Party that it is ready for occupancy. Optionally, that electronic communications may also be sent to the Controlling Party. If there is not a party seeking to occupy the confined space, then the electronic communications is sent only to the Controlling Party. Optionally, that electronic communications may include information enabling the Initiating Party to unlock the door and enter the confined space.

One additional option for systems that use a treatment device that applies a material (e.g. fogging aerosol) to the Confined Space is to include a separate device that confirms that the materials put into the treatment device are appropriate to assure the killing of viruses or germs (or both) in the confined space. For example, the refill canister might contain an RFID chip or other signal emitting device that provides information on the source and content of the refill canister, which information would be transmitted to the computer which could verify that such source and content were appropriate. This would make it more difficult for someone to try to use the system but with materials that would not achieve the required treatment.

Yet another option would be to allow the Initiating Party and/or the Controlling Party to access the some or all of the prior history regarding treatment of the specific confined space. Optionally, the treatment device may be powered by its own battery when used in vehicles.

The system uses a sensor to determine whether or not a particular confined space is empty. As anyone skilled in the art will know, this can be achieved with various types of sensors including a motion sensor or a thermal sensor.

Embodiments of the method used to assure disinfection of the confined space include the following steps. First, the Controlling Party initiates the germicidal and virus killing treatment process by sending an electronic communication to the computer controlling the germicidal and virus killing treatment process to initiate the process for a particular confined space. The computer then sends an electronic communication to one or more sensors in the confined space to determine if the confined space is occupied. If the sensors indicate that the confined space is occupied, the computer then sends an electronic communications to the Controlling Party indicating that the confined space is occupied, at which point the Controlling Party must check the confined space and if it is not occupied, re-initiate the process.

If the sensors indicate that the confined space is not occupied, the computer sends an electronic communications to the doors to the confined space locking them and then upon confirmation that the doors are locked, an electronic communications to the treatment device to initiate the germicidal and virus killing treatment process. Upon completion of the germicidal and virus killing treatment process including any time for the germicidal and virus killing material to settle in the case of a fogging device, the treatment device sends an electronic communication to the computer indicating that the germicidal and virus killing treatment process has been completed.

Optionally, the treatment device may provide other information about the germicidal and virus killing treatment process such as the fogging materials used and the source of such fogging materials. Optionally, when the treatment device is a fogger, it may use one or more pumps or compressors. Optionally, the computer may query one or more sensors for information about the germicidal and virus killing treatment process that can be provided by such sensors, including but not limited to the humidification level achieved. Upon receipt of confirmation that the treatment cycle has been completed, the computer sends an electronic communication advising the party seeking to occupy the space that it is ready for occupancy. Optionally, that electronic communications may also be sent to the Controlling Party.

If there is not a party seeking to occupy the confined space, then the electronic communications is sent only to the Controlling Party. Optionally, that electronic communications may include information enabling the party seeking occupancy to unlock the door and enter the confined space. Optionally, the electronic communications may include information regarding the method that was used to sterilize the confined space.

When the treatment device is a fogging system that uses two pumps instead of one, the first pump would pump the fogging agent from the reservoir to the nozzle, and the second pump would pump the fogging agent from the nozzle into the confined space. Although we use the word pump, it will be recognized by those skilled in the art that this could be accomplished through an air compressor or other means for creating an aerosol out of a liquid and air, and that the invention is not limited to the use of a pump.

One additional option is to have separate devices or multiple ports on the device so that different areas could be separately treated. For example, in the case of a vehicle which uses a fogging device or other device that disperses an aerosol, it might have two nozzles—one for the area in which the driver sits and one for the passenger compartment.

Another additional option for systems that use a fogger as the preferred treatment device is to include a separate device that confirms that the materials put into the fogger are appropriate to assure treatment of the confined space. For example, the refill canister might contain an RFID chip or other electronic communications emitting device that provides information on the source and content of the refill canister, which information would be transmitted to the computer which could verify that such source and content were appropriate. Yet another option would be for the computer to include with the electronic communications sent to the Initiating Party an inquiry as to whether the Initiating Party wants further history as to the treatment of the specific confined space. If the Initiating Party responds yes, the system provides such information.

Another option to assure that the correct refill canister is being used is to equip the refill canisters with RFID chips or other chips that would be checked by the treatment device. If the check determined that the refill canister did not carry a chip that it was intended for the treatment device, the treatment device would not allow that refill canister to open to the treatment device. Yet another additional option is to have the computer set a locking code

### BRIEF DESCRIPTION OF THE FIGURES

Embodiments of the present invention are described in detail below with reference to the attached drawing figures wherein:

FIG. 1 is a block flow diagram of the three major components in the simplest embodiment of the invention.

FIG. 2 is a block flow diagram of the system components in a more elaborate embodiment of the invention. It shows the components of such a system and how they connect to each other.

FIG. 3 is a block flow diagram of a simple embodiment of the method for use by taxi and car service drivers and companies.

FIG. 4 is a block flow diagram of a simple embodiment of the invention more suitable for vehicle rental companies and space rental companies such as hotels, daily office rentals and similar rental opportunities.

FIG. 5 is a block flow diagram of a more complete embodiment of the invention suitable for vehicle rental companies and space rental companies such as hotels, daily office rentals and similar rental opportunities.

### DETAILED DESCRIPTION

As used herein, certain terms are defined to clarify the intended meanings of those terms. These definitions apply regardless of whether or not the terms are capitalized.

As used herein, “communications device” and “electronic device” mean any device capable of two way electronic communications via a wireless network including but not limited to devices such as a cell phone, a general purpose computer, a wearable electronic device that can send and receive communications, special purpose programmable device or other commonly available communications device and that the controls for that device could be through an App that is downloaded to the device before the system is used. The computer could also be an App or program on a general purpose computer or laptop or iPad type device or even another communications device.

As used herein, the term computer is defined to refer includes, in addition to general purpose computers, programmable special purpose devices, simple programmable devices with the ability to process a limited set of instructions and communications as well as devices that have some or all of the functionality of a computer.

As used herein, the term treatment device includes any device that is capable of delivering a germicidal and virus killing treatment device to a confined space. By way of example and not limitation, the treatment device could be an ultraviolet light system such as what is disclosed in U.S. Pat. No. 8,680,496 or another form of ultraviolet system. It could also be a fogging or electrostatic spray device such as those offered by a wide variety of manufacturers under the names Goodway, Xpower, and Smartfog together with a disinfectant aerosol that appears on the EPA's list N (for example peroxycarboxylic acid or hydrogen peroxide).

As used herein in reference to this invention, the terms sanitizing and disinfecting and all forms of those verbs means the application of a germicidal and virus killing treatment to the space being treated.

The following detailed description of the invention references the accompanying drawings that illustrate specific embodiments in which the invention can be practiced. The embodiments are intended to describe aspects of the invention in sufficient detail to enable those skilled in the art to practice the inventions. Other embodiments can be utilized and changed without departing from the scope of the present invention. The following detailed description is, therefore, not to be taken in a limiting sense. The scope of the present invention is defined only by the appended claims, along with the full scope of equivalents to which such claims are entitled.

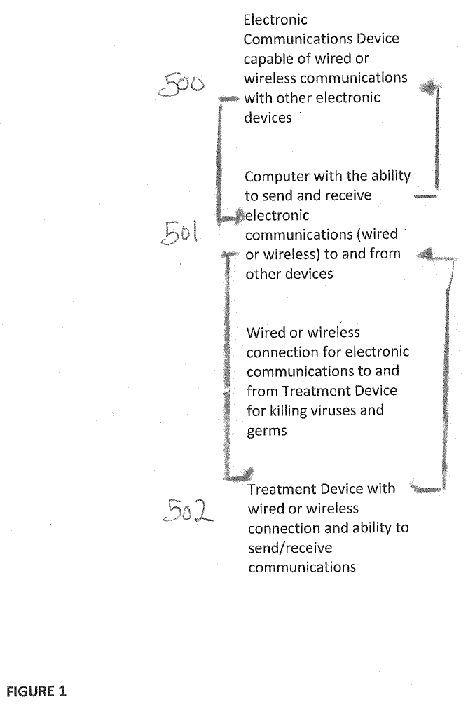

Turning now to FIG. 1, a simple embodiment of the germ and virus killing system that reports completion of its treatment process to the Initiating Party is disclosed. The simple embodiment of this system consists of an electronic device (100) that is capable of wired or wireless communications and can be used by an Initiating Party or the Controlling Person acting on behalf of a potential occupant to initiate a virus and germ killing treatment. The second component of this embodiment is a computer or programmable device (101) that processes the request and sends an electronic communications to a treatment device either located in or with access to the space instructing it to initiate the treatment. The third component is the treatment device (102) that delivers a germ and virus killing treatment to the space in question.

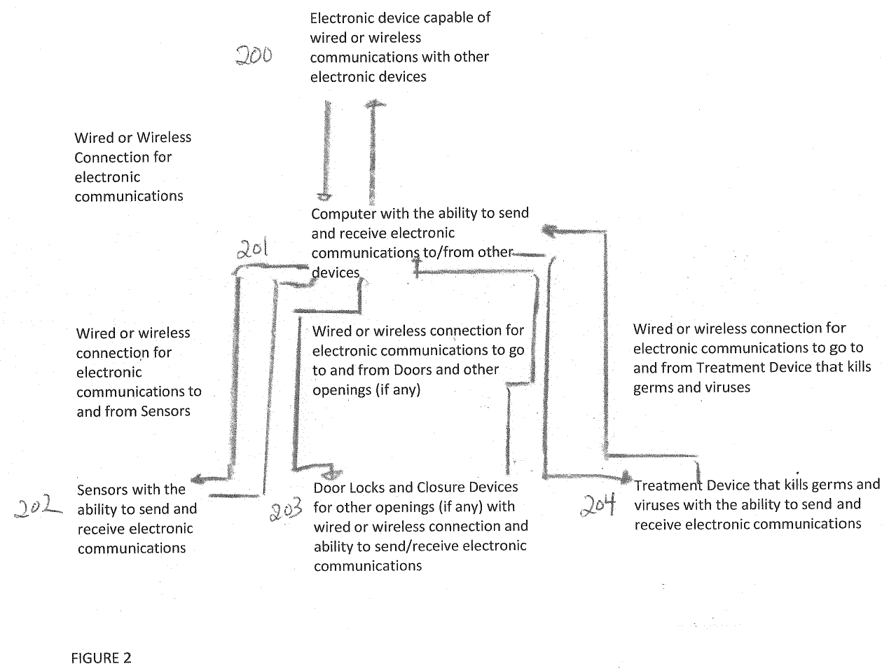

FIG. 2 is an embodiment that has an electronic communications device (200), a computer (201) and a treatment device (203) just like the embodiment in FIG. 1. It adds two additional components: sensors (202) that have the ability to determine whether or not a person is in a given space, such as motion detectors, infrared sensors or heat sensors and are further able to accept instruction from the computer and return information as to occupancy to the computer; and door locks (203) that are able to accept instruction from the computer as to when to unlock or lock the door and communicate the status of the door lock (open or shut) to the computer. Such additional components are particularly useful in facilities with multiple available spaces such as hotels and vehicle rental fleets.

The sensors allow the computer to determine whether or not the space is occupied before instructing the treatment device to initiate treatment of the space. By giving control over the door locks to the computer, the computer can prevent someone from inadvertently opening the door to the room while treatment is in process or after it is completed and before the scheduled occupant arrives.

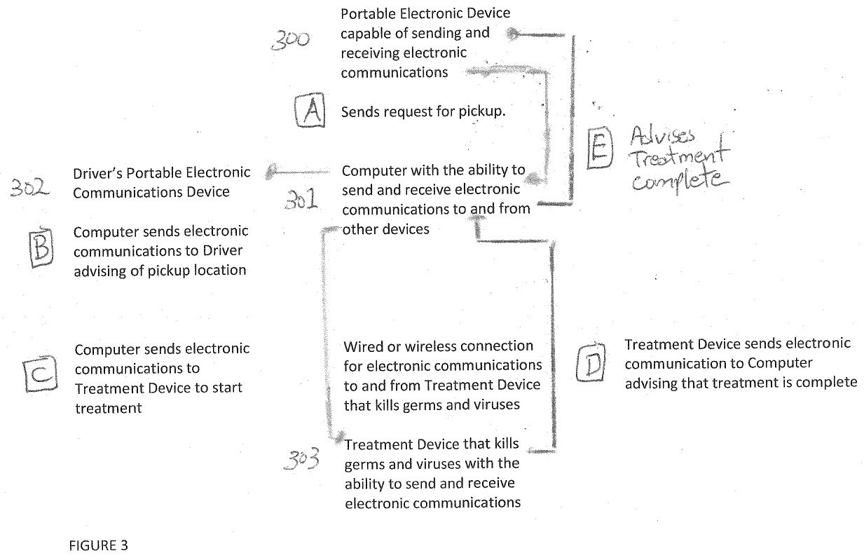

FIG. 3 is the simplest embodiment of the method for treating a confined space and informing the Initiating Party that it has been treated. This simple version is designed for taxis and car service vehicles, and contemplates that the process could start either because a person sends a request for a pickup to the computer (FIG. 3, A). Assuming that the vehicle is available, the computer then sends a message to the driver (B) as to the location of the pickup, and optionally a message to the person confirming the pickup (not shown). The computer then sends a message to the treatment device instructing it to begin the treatment of the confined space (C) and the treatment device does so.

When it is completed, the treatment device sends a message back to the computer that the treatment is complete (D). The computer then sends an electronic communications to the person who made the request advising that the treatment is complete and optionally that no one has been allowed to enter the confined space since the treatment was completed. As an additional option, the computer may also provide the person with details of the treatment.

This simple embodiment of the method is suitable to individual drivers who wish to offer potential passengers some assurance that the space in their vehicle has been disinfected. It may also be used by a driver who is flagged down by a potential passenger and enters the passenger data through the driver's portable electronic communications device, if the passenger is willing to allow the treatment cycle to run its course. This embodiment also works whether the entire interior of the vehicle including the driver's space is to be treated (although the driver would need to stop the vehicle in that case to allow the treatment cycle to complete) or if the passenger compartment is sealed off and treated during the treatment cycle.

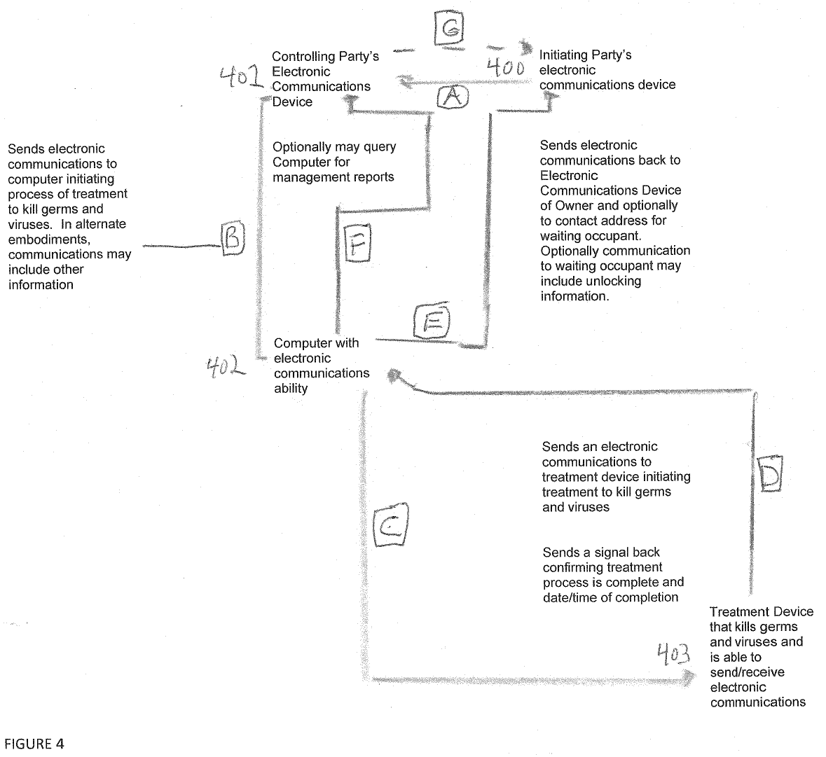

FIG. 4 is the simplest embodiment of the method for treating a confined space that is suitable for organizations that have fleets of vehicles or many confined spaces such as hotel rooms or offices available for daily or short term rental. This method begins when an Initiating Party using an electronic communications device (400) contacts the party that controls the confined space seeking to rent a confined space (FIG. 4, A). The Controlling Party (e.g. hotel reservation system or front desk) checks its inventory and if it has an available space, it proceeds to obtain information from the prospective occupant including an electronic contact address such as an email address or a location address if the prospective occupant uses an app to contact the Controlling party.

The Controlling party transmits the request, a proposed space identifier (e.g. room number) and contract information for the proposed occupant to a computer (B, 402). In this embodiment, the computer then transmits an instruction to the treatment device (403) in the confined space with that space identifier to begin the treatment (C). Upon completion of the treatment, the treatment device sends a communication back to the computer advising it that the treatment cycle has been completed (D). The computer then sends a communication to the Initiating Party advising the Initiating Party that the room is ready for occupancy. Optionally, the computer also sends that same information to the Controlling Party's electronic communications device (F).

In this version, it is assumed that a member of the Controlling Party's staff, in the case of a space rental and the service personnel, in the case of a returned rental vehicle, upon completion of the preparation work for occupancy that must be done by a person (e.g. empty wastebaskets, change linens and towels, check for items left behind) is completed and the Controlling Party is notified before it considers a room or vehicle to be available to be assigned and for treatment to begin.

It is also assumed that the housekeeping personnel, in the case of a room, and the service personnel, in the case of a vehicle, have locked the vehicle and placed the key or access card in a place where it can be obtained by the Initiating Party. These steps are not integral to the claimed invention of treating and communicating the completion of that treatment to the Initiating Party and therefore are not included as part of the claimed method.

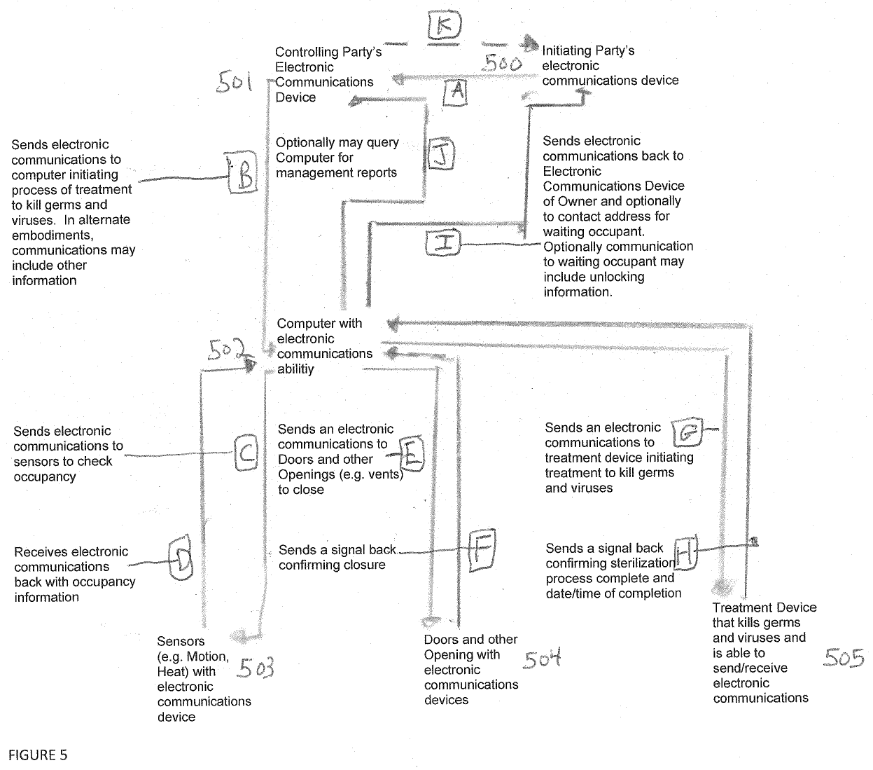

FIG. 5 is an enhanced embodiment of the method for treating a confined space that is suitable for organizations that have fleets of vehicles or many confined spaces such as hotel rooms or offices available for daily or short term rental. This embodiment starts the same as the prior embodiment, with an electronic communications device (500) contacts the party that controls the confined space seeking to rent a confined space (FIG. 5, A). The Controlling Party (e.g. hotel reservation system or front desk) checks its inventory and if it has an available space, it proceeds to obtain information from the prospective occupant including an electronic contact address such as an email address or a location address if the prospective occupant uses an app to contact the Controlling Party.

The Controlling Party transmits the request, a proposed space identifier (e.g. room number) and contract information for the proposed occupant to a computer (B, 502). In this embodiment, the computer then transmits an instruction to one or more sensors (503) in the confined space to determine whether or not a person is in the room (C). The sensor(s) then send back a communication. If the room is unoccupied, the computer proceeds with the process. If the room is occupied, the computer sends a request to the Controlling Party for a new room to be assigned and the process repeats itself. If the sensor reports that the room is not occupied, the computer sends a communication to one or more door locks (504) instructing them to set themselves in the locked position and to send back a communications once the doors are locked (E).

Upon receipt of confirmation that the doors are locked, the computer sends a communication to the treatment device (505) in the confined space with that space identifier to begin the treatment (G). Upon completion of the treatment, the treatment device sends a communication back to the computer advising it that the treatment cycle has been completed (H). The computer then sends a communication to the Initiating Party advising the Initiating Party that the room is ready for occupancy (I).

Optionally, the computer also sends that same information to the Controlling Party's electronic communications device (J). As an additional option, the message to the Initiating Party may include information as to a lock code or code for a box that holds the key card or key, so that the Initiating Party does not need to stop at a desk to check in. Optionally, the room may also have a device that shuts off internal air flow (heat or air conditioning duct or opening in the case of a car) and return duct (in the case of a room) that are controlled by an electronic communications device that is capable of communicating with the computer. Optionally, the sensors may also include a sensor that measures humidification to determine the materials delivered.

In such an option, the instruction to lock doors would also include an instruction to such vents to close the ducts, and the computer would send a communications to such ducts after the treatment cycle is completed allowing them to re-open. In this version, it also is assumed that a member of Controlling Party's staff, in the case of a space rental and the service personnel, in the case of a returned rental vehicle, upon completion of the preparation work for occupancy that must be done by a person (e.g. empty wastebaskets, change linens and towels, check for items left behind) is completed and the Controlling Party is notified before it considers a room or vehicle to be available to be assigned and for treatment to begin.

This step is not integral to the claimed invention of treating and communicating the completion of that treatment to the Initiating Party and therefore are not included as part of the claimed method.

Having thus described various embodiments of the invention, what is claimed as new, novel and desired to be protected by Letters Patent includes the following:

1. A method for treating a sealed passenger compartment of a vehicle with a germ and virus killing treatment and communicating the completion of that treatment to a person waiting to occupy the passenger compartment comprising the following steps:
A computer associated with a vehicle for hire that has an empty passenger space that is sealed off from the space occupied by the driver receiving a request via electronic communications from a person to pick up and take that person from one location to another;
The computer sending a communication to the person who initiated the request confirming the vehicle's availability and a communication to the driver advising the driver of the pickup location;
The computer sending an electronic instruction to a means for treating a passenger space with a germ and virus killing treatment that is capable of receiving and sending electronic communications to initiate the germ and virus killing treatment;
The means for treating a passenger space with a germ and virus killing treatment initiating the treatment and sending an electronic communication to the computer upon completion of the treatment confirming completion of the treatment;
The computer sending an electronic communication to the person who initiated the request confirming that the treatment was completed and that the person can enter the vehicle.

2. The invention of claim 1 wherein the passenger door locks are capable of electronic communications and of being opened and closed by such electronic communications and wherein the computer instructs the door locks to lock prior to initiating treatment; and wherein the computer sends to unlock code for the door locks to the person who initiated the request.

3. A method for treating a confined space with a germ and virus treatment and communicating to a person seeking to rent that the confined space that the confined space has been treated and is ready for occupancy comprising the following steps:
A computer receiving an inquiry from a person as to the availability of a confined space for rent and occupancy together with an electronic address to use for that person;
Identifying an appropriate confined space that has been cleaned and the door has been locked, but has not received a germ and virus killing treatment;
Sending an electronic communications to a means for killing viruses and germs that is located with access to such confined space and has the ability to receive and send electronic communications instructing it to begin a virus and germ killing treatment of the confined space;
The means for killing viruses and germs applying the treatment for killing viruses and germs to the confined space;
The means for killing viruses and germs sending an electronic communication to the computer upon completion of the treatment of the confined space indicating that the treatment has been completed;
The computer sending an electronic communication to the person advising the person that the treatment cycle has been completed and that the confined space is ready for occupancy.

4. The method of claim 3 wherein the electronic communication telling the person that the door has been treated also including information to enable the person to unlock the door and enter the confined space.

5. The method of claim 4 wherein the confined space is a room or vehicle available for rental such as a hotel room, automobile rental or office space rental.

6. The method of claim 4 wherein the confined space is the back passenger compartment of a vehicle for hire such as a tax or car service.

7. The method of claim 4 wherein the virus and germ killing treatment is initiated before any request is made for the confined space and the confined space is kept locked until a person seeks to occupy the confined space.

8. A method for treating a confined space to kill germs and viruses and communicating to a person that the confined space has been treated and is ready for occupancy comprising the following steps:
A computer receiving an inquiry from a person as to the availability of a confined space for occupancy together with an electronic address of the person;
The computer checking a database it maintains for available confined spaces and identifying a confined space that is ready for occupancy except that it has not yet received a virus and germ killing treatment;
The computer sending an electronic communication to at least one sensor in the confined space that is capable of sensing whether or not a person is present in the confined space instructing it to check the space for the presence of a person;
The sensor checking the confined space and sending an electronic communication back to the computer with the results of its check;
The computer reinitiating the process if the sensor's electronic communication indicates that the confined space is occupied;
The computer continuing the process if the confined space is unoccupied by sending an electronic communications to at least one door lock that is capable of communicating with the computer instructing it to lock the door if it is not already locked;
Upon locking the door, the door lock sending an electronic communication back to the computer confirming that the door is locked;
The computer sending an electronic communication to a treatment device to initiate treatment of the confined space;
The treatment device performing a germ and virus killing treatment on the confined space and sending an electronic communication to the computer when such treatment is completed;
Upon receipt of confirmation that the treatment cycle has been completed, the computer sending an electronic communications to the electronic address provided by the person seeking to occupy the confined space providing a description of the treatment applied, sending a signal that the treatment has been completed and identifying when it was completed.

9. The method of claim 8 wherein the virus and germ killing treatment is initiated before any request is made for the confined space and the confined space is kept locked until a person seeks to occupy the confined space.

10. The method of claim 9 wherein the person seeking to occupy the confined space is also told when the treatment was completed.

11. A system for treating a confined space to kill germs and viruses in the confined space and communicating with a person seeking to occupy that confined space that the treatment has been applied, comprising:
An electronic communications device capable of sending and receiving electronic communications;
A computer with the ability to send and receive electronic communications; and
A means for treating a confined space with a germ and virus killing treatment that is capable of receiving instructions from and sending information to the computer through electronic communications.

12. A system for treating a confined space to kill germs and viruses in the confined space and communicating with a person seeking to occupy the confined space that the treatment has been applied, comprising:
A computer with the ability to communicate with other electronic devices;
One or more sensors in a confined space with the ability to determine whether a person is occupying the confined space and to communicate with the computer;
One or more electronically controlled door locks for the confined space with the ability to communicate with the computer and accept instructions from the computer for locking and unlocking the one or more door locks;
A means for treating the confined space with a germ and virus killing treatment that is capable of receiving instructions from and sending information to the computer; and
An electronic device capable of receiving a communication advising it that the confined space has been disinfected and is ready for occupancy.
